SEQUENCE LISTING (4 sequences)

SEQ ID NO: 1 - protein, 593 aa, human
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - DNA, 1782 bp, human
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 3 - protein, 589 aa, Mus musculus
MWVLMSWLAFAAGLVAGTQCPDGQFCPVACCLDQGGANYSCCNPLLDTWPRITSHHLDGSCQTHGHCPAGYSCLLTVSGTSSCCPFSKGVSCGDGYHCCPQGFHCSADGKSCFQMSDNPLGAVQCPGSQFECPDSATCCIMVDGSWGCCPMPQASCCEDRVHCCPHGASCDLVHTRCVSPTGTHTLLKKFPAQKTNRAVSLPFSVVCPDAKTQCPDDSTCCELPTGKYGCCPMPNAICCSDHLHCCPQDTVCDLIQSKCLSKNYTTDLLTKLPGYPVKEVKCDMEVSCPEGYTCCRLNTGAWGCCPFAKAVCCEDHIHCCPAGFQCHTEKGTCEMGILQVPWMKKVIAPLRLPDPQILKSDTPCDDFTRCPTNNTCCKLNSGDWGCCPIPEAVCCSDNQHCCPQGFTCLAQGYCQKGDTMVAGLEKIPARQTTPLQIGDIGCDQHTSCPVGQTCCPSLKGSWACCQLPHAVCCEDRQHCCPAGYTCNVKARTCEKDVDFIQPPVLLTLGPKVGNVECGEGHFCHDNQTCCKDSAGVWACCPYLKGVCCRDGRHCCPGGFHCSARGTKCLRKKIPRWDMFLRDPVPRPLL

SEQ ID NO: 4 - DNA, 1770 bp, Mus musculus
atgtgggtcctgatgagctggctggccttcgcggcagggctggtagccggaacacagtgtccagatgggcagttctgccctgttgcctgctgccttgaccagggaggagccaactacagctgctgtaaccctcttctggacacatggcctagaataacgagccatcatctagatggctcctgccagacccatggccactgtcctgctggctattcttgtcttctcactgtgtctgggacttccagctgctgcccgttctctaagggtgtgtcttgtggtgatggctaccactgctgcccccagggcttccactgtagtgcagatgggaaatcctgcttccagatgtcagataaccccttgggtgctgtccagtgtcctgggagccagtttgaatgtcctgactctgccacctgctgcattatggttgatggttcgtggggatgttgtcccatgccccaggcctcttgctgtgaagacagagtgcattgctgtccccatggggcctcctgtgacctggttcacacacgatgcgtttcacccacgggcacccacaccctactaaagaagttccctgcacaaaagaccaacagggcagtgtctttgcctttttctgtcgtgtgccctgatgctaagacccagtgtcccgatgattctacctgctgtgagctacccactgggaagtatggctgctgtccaatgcccaatgccatctgctgttccgaccacctgcactgctgcccccaggacactgtatgtgacctgatccagagtaagtgcctatccaagaactacaccacggatctcctgaccaagctgcctggatacccagtgaaggaggtgaagtgcgacatggaggtgagctgccctgaaggatatacctgctgccgcctcaacactggggcctggggctgctgtccatttgccaaggccgtgtgttgtgaggatcacattcattgctgcccggcagggtttcagtgtcacacagagaaaggaacctgcgaaatgggtatcctccaagtaccctggatgaagaaggtcatagcccccctccgcctgccagacccacagatcttgaagagtgatacaccttgtgatgacttcactaggtgtcctacaaacaatacctgctgcaaactcaattctggggactggggctgctgtcccatcccagaggctgtctgctgctcagacaaccagcattgctgccctcagggcttcacatgtctggctcaggggtactgtcagaagggagacacaatggtggctggcctggagaagatacctgcccgccagacaaccccgctccaaattggagatatcggttgtgaccagcataccagctgcccagtagggcaaacctgctgcccaagcctcaagggaagttgggcctgctgccagctgccccatgctgtgtgctgtgaggaccggcagcactgttgcccggccgggtacacctgcaatgtgaaggcgaggacctgtgagaaggatgtcgattttatccagcctcccgtgctcctgaccctcggccctaaggttgggaatgtggagtgtggagaagggcatttctgccatgataaccagacctgttgtaaagacagtgcaggagtctgggcctgctgtccctacctaaagggtgtctgctgtagagatggacgtcactgttgccccggtggcttccactgttcagccaggggaaccaagtgtttgcgaaagaagattcctcgctgggacatgtttttgagggatccggtcccaagaccgctactgtaa

(19) United States
(12) Patent Application Publication
(10) Pub. No.: US 2013/0210039 A1
(43) Pub. Date: Aug. 15, 2013

(54) BLOOD INSULIN RESISTANCE AND DIABETES MARKER PROGRANULIN, METHOD FOR ANALYZING CONCENTRATION OF PROGRANULIN IN BLOOD SAMPLE, AND METHOD FOR SCREENING FOR SUBSTANCE THAT IMPROVES INSULIN RESISTANCE AND IMPROVES OR SUPPRESSES DIABETES

(71) Applicants: Toshiya Matsubara, Nishinomiya-shi (JP); Makoto Watanabe, Osaka-shi (JP); Susumu Seino, Ashiya-shi (JP); Kohtaro Minami, Kyoto-shi (JP); Osamu Nishimura, Kawanishi-shi (JP); Ayako Mita, Kobe-shi (JP)

(72) Inventors: Toshiya Matsubara, Nishinomiya-shi (JP); Makoto Watanabe, Osaka-shi (JP); Susumu Seino, Ashiya-shi (JP); Kohtaro Minami, Kyoto-shi (JP); Osamu Nishimura, Kawanishi-shi (JP); Ayako Mita, Kobe-shi (JP)

(21) Appl. No.: 13/819,704

(22) PCT Filed: Jul. 13, 2011
(86) PCT No.: PCT/JP2011/065993

§ 371 Date: Mar. 1, 2013

(30) Foreign Application Priority Data
Sep. 1, 2010 (JP) 2010-195871

(51) Int. Cl. G01N 33/68

(52) U.S. Cl. CPC G01N 33/6893
USPC 435/7.92; 530/326; 530/325; 530/324; 530/350

(57) ABSTRACT

The present invention provides a marker capable of detecting insulin resistance and diabetes in a collected blood sample, a method for analyzing said marker, a method for screening a substance improving insulin resistance, and improving or suppressing diabetes. A blood insulin resistance marker and a blood diabetes marker, which comprises a polypeptide comprising at least 15 continuous amino acids in an amino acid sequence constituting progranulin. A method for analyzing a blood marker, which comprises the steps of: measuring a concentration of an insulin resistance marker or a diabetes marker in a collected blood sample; and comparing the measured concentration with a normal blood concentration of the marker. A method for screening a substance improving insulin resistance, and improving or suppressing diabetes, which comprises the steps of: administering a candidate substance to a living body expressing insulin resistance or suffering from diabetes; measuring a blood concentration of a marker in the living body after administration; and comparing the measured concentration of the marker with a blood concentration of the marker not administered with the candidate substance.

### TECHNICAL FIELD

The present invention relates to the field of clinical diagnosis of insulin resistance and diabetes and the field of drug discovery searching for a substance that improves insulin resistance and diabetes.

### BACKGROUND ART

Diabetes is a disease having dysregulation of glucose metabolism in the body caused by reduced secretion of insulin from pancreatic β cells and impaired insulin action (insulin resistance) in liver, adipose tissue, skeletal muscle.

### (Diagnosis of Diabetes)

Currently, a blood glucose evaluation based on blood glucose level or hemoglobin Alc (HbAlc) level is used as the most common diagnostic criterion for diabetes diagnosis. According to diagnostic criteria defined by the Japan Diabetes Society, a fasting blood glucose level of lower than 110 mg/dL or a casual blood glucose level of lower than 140 mg/dL is considered as normal, a fasting blood glucose level of 126 mg/dL or higher or a casual blood glucose level of 200 mg/dL or higher is considered as diabetes, and a level outside the above ranges is considered as borderline. Such an evaluation based on blood glucose level is repeated, and when any of the evaluations is considered as diabetes, a diagnosis of diabetes is given. HbAlc is a glycosylated protein bound to excess glucose in blood, and its blood concentration is effective for an evaluation of a constant blood glucose level. Therefore, a diagnosis of diabetes is given by only a single evaluation based on constant blood glucose level when an HbAlc level is 6.5% or higher.

### (Treatment of Diabetes)

On the other hand, in the treatment of diabetes, there are known an insulin secretagogue that directly acts on pancreatic β cells to promote the secretion of insulin, biguanide that mainly acts on the liver to suppress gluconeogenesis, an a glucosidase inhibitor that inhibits absorption of glucose from the gastrointestinal tract, and a drug that improves insulin responsiveness. Further, in recent years, second-generation drugs such as a GLP-1 receptor agonist incretin that enhances the functions of gastrointestinal hormones to increase insulin secretion and DPP-4 inhibitors have come into use for diabetes treatment.

Among them, a drug that improves insulin sensitivity, that is, an “insulin resistance improving drug” is typified by a nuclear receptor (PPARγ)-targeting thiazolidine-based drug. Such a thiazolidine-based drug has the effect of enhancing insulin action in adipose tissue, liver, and muscle to normalize glucose metabolism in the body. An adipose tissue that mainly expresses PPARγ is an important endocrine organ that regulates insulin sensitivity in the body, and expresses and secretes cytokines (adipokines), such as adiponectin, leptin, and tumor necrosis factor α (TNF-α), which influence on differentiation or enlargement of adipose cells, insulin sensitivity, and the like. Further, it has been reported that enlargement of adipocytes due to obesity or the like causes abnormal production and section of adipokines and contributes to the development of insulin resistance (Non-Patent Document 1). PPARγ is a transcription factor that regulates differentiation or hypetrophy of adipocytes, and therefore it is considered that a thiazolidine-based drug acts on PPARγ in an adipose tissue to normalize the production and secretion of adipokines so that insulin resistance is improved.

As an experimental case using a thiazolidine-based drug to improve diabetes, the following has been reported. That is, 10-week-old diabetic mice were orally administered with a thiazolidine derivative (pioglitazone) every day for 1 week, and an oral glucose tolerance test (OGTT) was performed on these mice and a control group that was administered with only a solvent. As a result, the pioglitazone administration group had significantly low casual blood glucose levels (at 0 min) and blood glucose levels after a glucose load, that is, diabetes was improved (Non-Patent Document 6).

### (Detection of Insulin Resistance)

Insulin resistance can be considered as a first stage in the development of type II diabetes and is developed before a diagnosis of diabetes is given by a blood glucose evaluation. That is, during the first stage, an increase in insulin secretion from β cells compensates for a reduction in insulin responsiveness (insulin resistance) in muscle and liver so that the blood glucose level of a patient is kept normal (hyperinsulinemia). In the late stage of development of type II diabetes, β cell function is depressed so that impaired glucose tolerance is caused, and finally diabetes manifests itself to such an extent that a diagnosis of diabetes is given by the blood glucose evaluation. It has been demonstrated that early intervention with weight loss, exercise, or drug treatment can delay or prevent the development of diabetes in patients with impaired glucose tolerance (Non-Patent Document 4).

On the other hand, the most reliable method for detecting insulin resistance is an euglycemic-hyperinsulinemic clamp (EHC) method. Further, HOMA modeling is also often used as a diagnostic method for evaluating insulin resistance (Non-Patent Document 5). However, these methods take much time and labor and are therefore not suitable for diagnosis of a wide range of patients.

Therefore, effective biomarkers to detect insulin resistance have been reported (Patent Documents 1, 6, 7, 8, and 9).

### (Progranulin)

Progranulin is a granulin precursor protein having seven kinds of granulin domains. It has been reported that progranulin and granulins peptides have a growth factor-like action when secreted outside the cell, and are involved in inflammation and cell migration (Non-Patent Document 3, and Patent Documents 1, 2, 3, 4, and 5).

The relationship between progranulin and insulin resistance had not been elucidated, but the present inventors have identified, in cultured adipocytes, an insulin resistance marker progranulin (also known as Proepitherin/PGRN/PCDGF/PEPI/GEP/GP88) that can detect impaired insulin action and have elucidated the relationship between insulin resistance and progranulin expression (Patent Document 1).

Specifically, the expression level of progranulin was increased by treating cultured adipocytes with TNF-α or dexamethasone that indirectly impairs insulin action in cells. Further, it has been demonstrated that the increase in the expression level caused by impaired insulin action is completely suppressed by adding the above-described thiazolidine-based drug (pioglitazone) that improves insulin action. Further, the present inventors have elucidated that progranulin has the action of reducing insulin sensitivity of adipocytes. Specifically, the present inventors have elucidated that the activity of an Akt protein activated downstream of an insulin receptor is significantly suppressed under insulin stimulation by treating cultured adipose cells with a progranulin protein. Further, it has also been found that insulin responsiveness is improved by suppressing the expression of progranulin by infecting adipocytes with shRNA-expressing adenovirus targeting a progranulin gene.

### PRIOR ART DOCUMENTS

### Patent Documents

Patent Document 1: JP-A-2009-204475
Patent Document 2: JP-A-2006-513171
Patent Document 3: JP-A-2008-515459
Patent Document 4: JP-A-2009-109489
Patent Document 5: JP-A-2009-538631
Patent Document 6: JP-A-2004-041208
Patent Document 7: JP-A-2005-006645
Patent Document 8: JP-A-2008-523398
Patent Document 9: JP-A-2008-536474

### Non-Patent Documents

Non-Patent Document 1: Diabetes & Metabolism, 34 (2008) 2-11
Non-Patent Document 2: Molecular and Cellular Endocrinology, 316 (2010) 129-139
Non-Patent Document 3: J Mol Med 81 (2003) 600-612
Non-Patent Document 4: N. Engl. J. Med. 346 (2002) 393-403
Non-Patent Document 5: Diabetes Care 27 (2004) 1487-1495
Non-Patent Document 6: Journal of Biological Chemistry 281, 8748-8755

### SUMMARY OF THE INVENTION

### Problems to be Solved by the Invention

### (Diabetes Marker)

Real threats of diabetes are its complications, and examples of serious chronic complications include retinopathy, diabetic nephropathy, vascular disorders such as myocardial infarction and cerebrovascular diseases, and neuropathy. It is known that the risk of such complications is already increased before development of diabetes. Therefore, there is a demand for an early diagnostic marker for finding diabetes earlier and a method for preventing or improving diabetes earlier. A blood glucose evaluation based on repeated tests of blood glucose level or HbAlc level, which is now widely used, is effective for detection of a complete diabetic state with a sufficiently high blood glucose level, but has difficulty in early diagnosis or detection of borderline diabetes. Further, an evaluation of efficacy of diabetes drugs and determination of diabetes treatment strategies cannot be satisfactorily performed only by the blood glucose evaluation.

### (Insulin Resistance Marker)

It is estimated that early diagnosis of insulin resistance makes it possible to perform early intervention with anti-diabetic treatment or other methods for preventing the progression of diabetes. A marker for insulin resistance is very effective for detection of such a state. That is, it is expected that an appropriate insulin resistance marker will become a disease biomarker that makes it possible to achieve early diagnosis in, for example, borderline diabetes and before development of diabetes.

Diseases such as diabetes are, in fact, complex systemic diseases involving various predisposing factors in the body. Therefore, when an early diagnosis of diabetes is performed using an insulin resistance marker, a marker whose expression and secretion have merely been confirmed at a cellular level, that is, a marker intended to detect so-called local insulin resistance is not appropriate as the insulin resistance marker. For example, JP-A-2009-204475 (Patent Document 1) merely discloses that it has been confirmed that progranulin is expressed and secreted in cultured cells. Accordingly, it is merely suggested that progranulin is intended to detect local insulin resistance.

### (Method for Searching Novel Drug Target)

A thiazolidine derivative that is a drug for treating diabetes by improving insulin resistance has a strong ability to promote differentiation of adipose cells. Therefore, administration of the drug involves the risk of weight gain or obesity, and there are concerns about the safety of the drug due to such side effects. Accordingly, there is a demand for a method for searching a novel drug candidate compound for improving insulin resistance.

It is an object of the present invention to provide a simple and accurate clinical diagnostic marker capable of evaluating insulin resistance.

It is an object of the present invention to provide not a marker whose expression and secretion are confirmed in cells caused with impaired insulin action, that is, which is intended to detect so-called cellular insulin resistance but a marker that is expressed and secreted in a living body having insulin resistance symptoms and is capable of detecting so-called systemic insulin resistance.

It is an object of the present invention to provide a marker capable of detecting diabetes.

It is an object of the present invention to provide a blood marker analysis method capable of examining insulin resistance by measuring the concentration of a marker in a collected blood sample.

It is an object of the present invention to provide a blood marker analysis method capable of examining diabetes by measuring the concentration of a marker in a collected blood sample.

It is an object of the present invention to provide a method for searching a novel drug candidate compound for improving insulin resistance.

It is an object of the present invention to provide a method for searching a novel drug candidate compound for improving diabetes.

### Means for Solving the Problems

The present inventors have found that the above objects of the present invention can be achieved by progranulin, which has led to the completion of the present invention.

The present invention includes the following aspects.

A “polypeptide” used as a marker according to the present invention includes a polypeptide specified by a specific sequence (i.e., a sequence comprising at least 15 continuous amino acids in an amino acid sequence represented by SEQ ID No. 1) and polypeptides that are homologs and mutants of the above polypeptide and have an equivalent biological function involved in insulin resistance to that of the above polypeptide.

Therefore, the “polypeptide comprising at least 15 continuous amino acids in an amino acid sequence represented by SEQ ID No. 1” includes, as one example, a polypeptide comprising at least 15 continuous amino acids in an amino acid sequence represented by SEQ ID No. 3.

The “polypeptide” includes an oligopeptide and a protein.

(1) A blood insulin resistance marker comprising a polypeptide comprising at least 15 continuous amino acids in an amino acid sequence represented by SEQ ID No. 1.

The “insulin resistance” refers to a state in which a normal physiological or molecular response cannot be generated by a normal amount of insulin. In some cases, a state may be included, in which at least part of a normal physiological or molecular response can be improved or a biological response can be generated by an excess physiological amount of insulin endogenously produced or externally added. The “insulin resistance” in the present invention refers to not a state of impaired insulin action observed at a cellular level but so-called systemic insulin resistance that is a state in which the physiological action of insulin on glucose metabolism, lipid metabolism in a living body is significantly impaired.

The “insulin resistance marker” refers to a marker for evaluating systemic insulin resistance. The “evaluation of insulin resistance” includes identifying a general state with insulin resistance and identifying the affected state of a disease with insulin resistance, and more specifically includes performing detection and diagnosis of insulin resistance, and detection, diagnosis, monitoring, staging, and prognosis of a disease with insulin resistance.

(2) A method for analyzing an insulin resistance marker in a collected blood sample, comprising the steps of:

measuring a concentration of a polypeptide comprising at least 15 continuous amino acids in an amino acid sequence represented by SEQ ID No. 1 in a collected blood sample; and

comparing the obtained measured concentration with a normal blood concentration of the polypeptide.

The blood sample is collected from a living body to be evaluated for insulin resistance. An increase in the measured concentration of the polypeptide in the blood sample above the normal blood concentration of the polypeptide can be regarded as one index indicating that the living body has a high possibility of being in an insulin-resistance state.

(3) A method for screening a substance improving insulin resistance, comprising the steps of:

administering a candidate substance to a non-human animal showing abnormal insulin sensitivity;

measuring a blood concentration of a polypeptide comprising at least 15 continuous amino acids in an amino acid sequence represented by SEQ ID No. 1 in the non-human animal administered with the candidate substance; and

comparing the measured blood concentration of the polypeptide in the non-human animal administered with the candidate substance, with a blood concentration of the polypeptide in a non-human animal not administered with the candidate substance, wherein

a reduction in the concentration in the non-human animal administered with the candidate substance below the concentration in the non-human animal not administered with the candidate substance is regarded as one index for selecting the candidate substance as a substance improving insulin resistance.

A method for screening a substance improving insulin resistance, comprising the steps of:

administering a candidate substance to a living body showing abnormal insulin sensitivity;

measuring a blood concentration of a polypeptide comprising at least 15 continuous amino acids in an amino acid sequence represented by SEQ ID No. 1 in the living body administered with the candidate substance; and

comparing the measured blood concentration of the polypeptide in the living body administered with the candidate substance, with a blood concentration of the polypeptide in a living body not administered with the candidate substance, wherein

a reduction in the measured concentration in the living body administered with the candidate substance below the concentration in the living body not administered with the candidate substance is regarded as one index for selecting the candidate substance as a substance improving insulin resistance.

The “improvement of insulin resistance” includes changing insulin resistance to higher insulin sensitivity and changing insulin resistance to higher insulin-dependent glucose transport activity, and more specifically includes controlling an insulin signal transduction-suppressing action in insulin resistance and controlling an insulin-dependent glucose transport system-suppressing action, and an example thereof includes treating a disease with insulin resistance.

(4) A blood diabetes marker comprising a polypeptide comprising at least 15 continuous amino acids in an amino acid sequence represented by SEQ ID No. 1.

The “diabetes marker” refers to a marker for evaluating diabetes. The “evaluation of diabetes” includes identifying the affected state of diabetes, and more specifically includes performing prediction, detection, diagnosis, monitoring, staging, and prognosis of diabetes. The prediction of diabetes includes identifying borderline diabetes (early diagnosis of diabetes). The borderline diabetes includes an insulin-resistance state without hyperglycemia or an impaired glucose tolerance condition.

(5) A method for analyzing a diabetes marker in a collected blood sample, comprising the steps of:

measuring a concentration of a polypeptide comprising at least 15 continuous amino acids in an amino acid sequence represented by SEQ ID No. 1 in a collected blood sample; and

comparing the obtained measured concentration and a normal blood concentration of the polypeptide.

The blood sample is collected from a living body to be evaluated for diabetes. An increase in the measured concentration of the polypeptide in the blood sample above the normal blood concentration of the polypeptide can be regarded as one index indicating that the living body is predicted to develop diabetes or has a high possibility of being in a diabetic state.

(6) A method for screening a substance improving or suppressing diabetes, comprising the steps of:

administering a candidate substance to a non-human animal suffering from diabetes;

measuring a blood concentration of a polypeptide comprising at least 15 continuous amino acids in an amino acid sequence represented by SEQ ID No. 1 in the non-human animal administered with the candidate substance; and

comparing the measured blood concentration of the polypeptide in the non-human animal administered with the candidate substance, with a blood concentration of the polypeptide in a non-human animal not administered with the candidate substance, wherein

a reduction in the concentration in the non-human animal administered with the candidate substance below the concentration in the non-human animal not administered with the candidate substance is regarded as one index for selecting the candidate substance as a substance improving or suppressing diabetes.

A method for screening a substance improving or suppressing diabetes, comprising the steps of:

administering a candidate substance to a living body suffering from diabetes;

measuring a blood concentration of a polypeptide comprising at least 15 continuous amino acids in an amino acid sequence represented by SEQ ID No. 1 in the living body administered with the candidate substance; and

comparing the measured blood concentration of the polypeptide in the living body administered with the candidate substance, with a blood concentration of the polypeptide in a living body not administered with the candidate substance, wherein

a reduction in the measured concentration in the living body administered with the candidate substance below the concentration in the living body not administered with the candidate substance is regarded as one index for selecting the candidate substance as a substance improving or suppressing diabetes.

### Effects of the Invention

According to the present invention, it is possible to provide a simple and accurate clinical diagnostic marker capable of evaluating insulin resistance.

According to the present invention, it is possible to provide not a marker whose expression and secretion are confirmed in cells caused with impaired insulin action, that is, which is intended to detect so-called cellular insulin resistance, but a marker that is expressed and secreted in a living body having insulin resistance symptoms and is capable of detecting so-called systemic insulin resistance.

According to the present invention, it is possible to provide a marker capable of detecting diabetes.

According to the present invention, it is possible to provide a blood marker analysis method capable of examining insulin resistance by measuring the concentration of a marker in a collected blood sample.

According to the present invention, it is possible to provide a blood marker analysis method capable of examining diabetes by measuring the concentration of a marker in a collected blood sample.

According to the present invention, it is possible to provide a method for searching a novel drug candidate compound for improving insulin resistance.

According to the present invention, it is possible to provide a method for searching a novel drug candidate compound for improving diabetes.

### BRIEF DESCRIPTION OF THE DRAWINGS

FIG. 1 shows changes in blood glucose level in ob/ob mice (insulin-resistance model mice) and ob/+ mice (control mice).

FIG. 2 shows changes in blood insulin concentration in ob/ob mice (insulin-resistance model mice) and ob/+ mice (control mice).

FIG. 3 shows expression levels of progranulin (PGRN) mRNA in adipose tissues (epididymal fat tissue (EF), mesenteric fat tissue (MF), and subcutaneous adipose tissue (SC) (white adipose tissue (WAT) and brown adipose tissue (BAT)) of ob/ob mice (insulin-resistance model mice) and ob/+ mice (control mice).

FIG. 4 shows blood progranulin (PGRN) concentrations in ob/ob mice (insulin-resistance model mice) and ob/+ mice (control mice).

FIG. 5 shows changes in blood glucose level in db/db mice (diabetes model mice) and db/+ mice (control mice).

FIG. 6 shows changes in blood insulin concentration in db/db mice (diabetes model mice) and db/+ mice (control mice).

FIG. 7 shows blood progranulin (PGRN) concentrations in db/db mice (diabetes model mice) and db/+ mice (control mice).

FIG. 8 shows expression levels of progranulin (PGRN) mRNA in adipose tissues (epididymal fat tissue (EF), mesenteric fat tissue (MF), and subcutaneous adipose tissue (SC) (white adipose tissue (WAT) and brown adipose tissue (BAT)) of ob/ob mice administered with pioglitazone (insulin-resistance model mice administered with pioglitazone, Pio), ob/ob mice administered with only a solvent (insulin-resistance model mice administered with only a solvent, Vehicle), and ob/+ mice.

FIG. 9 shows blood progranulin (PGRN) concentrations in ob/ob mice administered with pioglitazone (insulin-resistance model mice administered with pioglitazone, Pio), ob/ob mice administered with only a solvent (insulin-resistance model mice administered with only a solvent, Vehicle), and ob/+ mice.

FIG. 10 shows body weight changes in knock-out mice lacking a progranulin gene (PGRN−/−) and wild-type mice (WT) fed a standard diet (SD) (a), body weight changes in knock-out mice lacking a progranulin gene (PGRN−/−) and wild-type mice (WT) fed a high-fat diet (HFD) (b), and food intake (c).

FIG. 11 shows the observation of visceral adipose accumulation in knock-out mice lacking a progranulin gene (PGRN−/−) and wild-type mice (WT) fed with standard diet (SD) a high-fat diet (HFD) (a), the observation of the shape of adipose cells by hematoxylin-eosin staining (HE) (b), and the observation of inflammatory cells by immunohistochemical staining using anti-Mac3 antibody (Mac3) (c).

FIG. 12 shows the results of an insulin tolerance test performed on knock-out mice lacking a progranulin gene (PGRN−/−) and wild-type mice (WT) fed a high-fat diet (HFD) and knock-out mice lacking a progranulin gene (PGRN−/−) and wild-type mice (WT) fed a standard diet (SD).

FIG. 13 shows the results of an oral glucose tolerance test performed on knock-out mice lacking a progranulin gene (PGRN−/−) and wild-type mice (WT) fed a high-fat diet (HFD), and (a) shows a change in blood glucose level and (b) shows a change in blood insulin concentration.

### MODES FOR CARRYING OUT THE INVENTION

### [1. Insulin Resistance Marker]

A marker according to the present invention shows specificity for insulin resistance. The “insulin resistance” refers to a state in which a normal physiological or molecular response cannot be generated by a normal amount of insulin. In some cases, a state may be included, in which at least part of a normal physiological or molecular response can be improved or a biological response can be generated by an excess physiological amount of insulin endogenously produced or externally added. The “insulin resistance” in the present invention refers to not a state of impaired insulin action observed at a cellular level but so-called systemic insulin resistance that is a state in which the physiological action of insulin on glucose metabolism, lipid metabolism, or the like in a living body is significantly impaired.

Here, a specific definition of “normal amount” is appropriately given by those skilled in the art based on a known method for evaluating insulin resistance. For example, in the case of an adult, blood insulin (IRI value) is 10 μU/mL or less during fasting. In the case of a mouse, a blood insulin concentration is, for example, about 1 ng/mL. However, the normal amount is not limited thereto and is defined by various other factors.

Further, a quantitative definition of “insulin resistance” is also appropriately given by those skilled in the art based on a known method for evaluating insulin resistance. For example, it is often the case that deviation from 2.5 or less, which is a normal HOMA-R value, is diagnosed as possibly having insulin resistance. In the case of a mouse, for example, the fasting blood insulin concentration of an ob/ob mouse with insulin resistance is 10 ng/mL or higher, whereas the fasting blood insulin concentration of an ob/+ mouse without insulin resistance is about 1 ng/mL. Further, the quantitative definition may be given based on blood glucose level because a change in blood glucose is markedly increased in an insulin tolerance test. However, the quantitative definition of insulin resistance is not limited thereto and may be defined by other factors.

An “insulin resistance marker” refers to a marker evaluating insulin resistance, and includes a marker identifying a condition with insulin resistance and a marker identifying the affected state of a disease with insulin resistance.

Examples of the condition with insulin resistance and the affected state of a disease with insulin resistance include Type II diabetes, metabolic syndrome, prediabetes, polycystic ovarian syndrome, abnormal lipid metabolism, obesity, infertility, inflammatory disorder, cancer, inflammatory disease, Alzheimer's disease, hypertension, atherosclerosis, cardiovascular disease, and peripheral vascular disease.

### [2. Diabetes Marker]

The marker according to the present invention is specific to insulin resistance, and is therefore useful as a marker for diabetes that is one of diseases showing insulin resistance. A “diabetes marker” as used in the present invention refers to a marker evaluating Type II diabetes, and includes a marker predicting diabetes and a marker identifying diabetes. The “prediction of diabetes” includes identifying borderline diabetes (early diagnosis of diabetes). The borderline diabetes includes an insulin-resistance state without hyperglycemia or an impaired glucose tolerance condition. The marker according to the present invention is specific to insulin resistance, and is therefore capable of detecting the early stage of diabetes without hyperglycemia (borderline diabetes), which is very advantageous in that early diagnosis of diabetes can be achieved.

### [3. Marker Component Polypeptide]

The blood insulin resistance marker and blood diabetes marker according to the present invention include a polypeptide comprising at least 15 continuous amino acids in an amino acid sequence represented by SEQ ID No. 1.

The “polypeptide” includes an oligopeptide and a protein.

The “amino acid sequence represented by SEQ ID No. 1” is a sequence of a human homologue of progranulin (full length).

The “polypeptide comprising at least 15 continuous amino acids in an amino acid sequence represented by SEQ ID No. 1” includes progranulin (full length), granulin peptides (specifically, granulin-1, granulin-2, granulin-3, granulin-4, granulin-5, granulin-6, and granulin-7), and any polypeptide containing a minimum sequence involved in insulin resistance in a progranulin sequence in its part or entirety. It is to be noted that a peptide corresponding to amino acids 284-335 of progranulin (full length) represented by SEQ ID No. 1 is granulin-1, a peptide corresponding to amino acids 208-260 of that is granulin-2, a peptide corresponding to amino acids 366-416 of that is granulin-3, a peptide corresponding to amino acids 443-494 of that is granulin-4, a peptide corresponding to amino acids 520-571 of that is granulin-5, a peptide corresponding to amino acids 126-178 of that is granulin-6, and a peptide corresponding to amino acids 61-112 of that is granulin-7.

The polypeptide comprising at least 15 continuous amino acids in an amino acid sequence represented by SEQ ID No. 1 may be a polypeptide comprising at least 50 continuous amino acids in the sequence or a polypeptide comprising at least 593 continuous amino acids in the sequence.

The “polypeptide comprising at least 15 continuous amino acids in an amino acid sequence represented by SEQ ID No. 1” includes a polypeptide specified by a sequence comprising at least 15 continuous amino acids in an amino acid sequence represented by SEQ ID No. 1 and polypeptides that are homologs and mutants of the polypeptide and have an equivalent biological function involved in insulin resistance to that of the polypeptide.

The above-described homologs include complete or partial sequences of homologs of other species corresponding to a human homolog of progranulin. Examples of the other species include mammals other than humans, and specific examples thereof include primates, rodents (e.g., mice and rats), rabbits, dogs, cats, pigs, cows, sheep, and horses.

Therefore, the “polypeptide comprising at least 15 continuous amino acids in an amino acid sequence represented by SEQ ID No. 1” includes a polypeptide comprising at least 15 continuous amino acids in an amino acid sequence represented by SEQ ID No. 3 that is a mouse homolog.

The above-described mutants include naturally-occurring mutants of progranulin and mutants modified by artificial replacement, addition, insertion, and deletion of an amino acid.

Examples of these homologs and mutants include those having a homology of at least 80%, preferably at least 85%, more preferably at least 90%, even more preferably at least 95% with the sequence comprising at least 15 continuous amino acids in an amino acid sequence represented by SEQ ID No. 1.

For example, it is well known that the amino acid homology between mouse and human is 84% or higher. It is generally recognized that metabolic mechanism and signal transduction mechanism that bear the fundamentals of life are extremely similar between mouse and human. In fact, those exhibiting equivalent functions and expression variations in adipose cells between mouse and human have been frequently reported, and therefore it is clear that glucose metabolism regulation and insulin signal transduction are no exception. It is commonly recognized among those skilled in the art that it is highly likely that glucose metabolism regulation, insulin signal transduction and the like are mechanisms common to all mammals including mice and humans. Therefore, it is very reasonable that a polynucleotide in the present invention includes one that basically has a sequence comprising at least 15 continuous amino acids in an amino acid sequence represented by SEQ ID No. 1 and has a homology of at least 80%. For example, a homology between a mouse homolog represented by SEQ ID No. 3 and a human homolog represented by SEQ ID No. 1 is 84% (by BLAST homology search).

### [4. Insulin Resistance Marker Analysis Method]

An evaluation of insulin resistance can be achieved by analyzing the blood concentration of the above-described insulin resistance marker according to the present invention.

The “evaluation of insulin resistance” includes identifying a general state with insulin resistance and identifying the affected state of a disease with insulin resistance, and more specifically includes performing detection and diagnosis of insulin resistance, and detection, diagnosis, monitoring, staging, and prognosis of a disease with insulin resistance. The prognosis includes, when treatment for improving the disease is performed, determining the presence or absence of improvement in the disease or the degree of the improvement after the treatment.

### [4-1. Processes of Insulin Resistance Marker Analysis]

A method for evaluating insulin resistance according to the present invention includes the steps of: measuring a concentration of the above-described polypeptide in a collected blood sample; and comparing the obtained measured concentration with a normal blood concentration of the above-described polypeptide.

An increase in the measured concentration of the above-described polypeptide in the collected blood sample above the normal blood concentration of the above-described polypeptide can be regarded as one index indicating that a living body has a high possibility of being in an insulin-resistance state.

In the method according to the present invention, the measured result of the polypeptide according to the present invention may be used singly or in combination with the measured result of any other marker related to the condition or disease state of insulin resistance. Therefore, the method according to the present invention may include measuring concentrations of other markers as well as a concentration of the polypeptide according to the present invention.

### [4-2. Collected Blood Sample]

In the present invention, a collected blood sample is used as an object to be analyzed. The blood sample is collected from a living body to be evaluated for insulin resistance. The collected blood sample may be subjected to any pretreatment that does not have essentially influence on analysis. Examples of such pretreatment include anticoagulation treatment and centrifugal separation (plasma separation and serum separation), but plasma exchange, serum exchange, and addition of a substance having the ability to bind to the polypeptide according to the present invention (by which the polypeptide according to the present invention may be eliminated from an analysis system) are excluded. Therefore, examples of the collected blood sample include whole blood, plasma, and serum.

The collected blood sample may be derived from any living body. The living body includes all animals including human. For example, mammals can be mentioned. Examples of the mammals include primates (e.g., humans), rodents (e.g., mice and rats), rabbits, dogs, cats, pigs, cows, sheep, and horses.

### [4-3. Method for Measuring Polypeptide Concentration]

A method for measuring a concentration of the polypeptide in a collected blood sample is not particularly limited, and any method capable of specifically detecting a specific polypeptide can be used. Specifically, it may be measured by a test based on biospecific affinity or by mass spectrometry.

The test based on biospecific affinity is a well-known method to those skilled in the art, and examples thereof include, but are not particularly limited to, immunoassays. Specific examples of the immunoassays include competitive and non-competitive assay systems such as Western blotting, radio immunoassay, ELISA, sandwich immunoassay, immunoprecipitation, precipitation reaction, gel diffusion precipitin reaction, immunodiffusion, aggregation measurement, complement fixation analysis, immunoradiometric assay, fluorescent immunoassay, and protein A immunoassay. In these methods, the presence of an antibody that binds to progranulin in a blood sample is detected. The test is performed, under the condition that an immunocomplex composed of a polypeptide to be measured and an antibody to the polypeptide can be formed, by bringing a blood sample into contact with the antibody. Amore specific protocol can be easily determined by those skilled in the art.

As a method measured by mass spectrometry, a method capable of quantitatively measuring a polypeptide can be appropriately selected by those skilled in the art.

For example, an isotope labeling method can be mentioned. The isotope labeling method is preferred because it is excellent in quantitativity. In this case, the measurement can be performed by determining a difference in the presence of the above-described polypeptide between an appropriate control sample, such as a sample prepared to contain a known level of the above-described polypeptide or a normal sample, and a sample of an individual to be analyzed.

Further, a method can be mentioned, in which an immunoprecipitate is obtained from a collected blood sample and subjected to mass spectrometry (WO 2008/065806). In this case, the polypeptide according to the present invention present in a collected blood sample is immunoprecipitated with a carrier having an antibody to the polypeptide bonded thereto or a carrier having the antibody and a molecule, which can specifically bind to the antibody, bonded thereto. The obtained polypeptide immunoprecipitate is washed with an aqueous solution containing a charge neutralizer (e.g., an ammonium salt such as ammonium carbonate or ammonium hydrogen carbonate). Then, the immunoprecipitate can be directly subjected to mass spectrometry to quantitatively measure the polypeptide based on an internal standard.

### [4-4. Evaluation Between Normal and Insulin Resistance]

The “normal concentration” of the polypeptide in blood includes the blood concentration of the polypeptide according to the present invention in an insulin-sensitive living body. The “insulin sensitivity” refers to a state in which a normal physiological or molecular response can be generated by a normal amount of insulin.

Here, a specific definition of “normal amount” is appropriately given by those skilled in the art based on a known method for evaluating insulin resistance. For example, in the case of an adult, blood insulin (IRI value) is 10 μU/mL or less during fasting. In the case of a mouse, a blood insulin concentration is, for example, about 1 ng/mL during fasting. However, the normal amount is not limited thereto and is defined by various other factors.

Further, a quantitative definition of “insulin resistance” is also appropriately given by those skilled in the art based on a known method for evaluating insulin resistance. For example, it is often the case that deviation from 2.5 or less, which is a normal HOMA-R value, is diagnosed as possibly having insulin resistance. In the case of a mouse, for example, the fasting blood insulin concentration of an ob/ob mouse with insulin resistance is 10 ng/mL or higher, whereas the fasting blood insulin concentration of an ob/+ mouse without insulin resistance is about 1 ng/mL. Further, the quantitative definition may be given based on blood glucose level because a change in blood glucose is markedly increased in an insulin tolerance test. However, the quantitative definition of insulin resistance is not limited thereto and may be defined by other factors.

Therefore, the normal blood concentration of the polypeptide according to the present invention may be the concentration of the polypeptide according to the present invention in a living body that should be judged to be normal based on a known insulin resistance evaluation method. Such a normal blood concentration can be measured by those skilled in the art, and the range of a normal blood concentration can be appropriately determined by those skilled in the art based on the measurement results and a known insulin resistance evaluation method. For example, in the case of rodents such as mice, a normal concentration may be 0.2 μM to 0.3 μM.

Further, the degree of an increase in the measured concentration of the polypeptide according to the present invention above the normal concentration by which a living body is judged to have a high possibility of being in an insulin-resistance state is not particularly limited. The measured concentration of the polypeptide according to the present invention, at which it is judged to have a high possibility of being in an insulin-resistance state can reach, for example, the concentration of the polypeptide according to the present invention in the blood of a living body that should be judged to be insulin resistant based on a known insulin resistance evaluation method.

For example, a degree such that the measured concentration is 1.25 times or more, preferably 1.5 times or more, more preferably 2 times or more (molar basis) the normal concentration can be regarded as a standard by which it is judged to be insulin resistant. For example, in the case of rodents such as mice, a measured polypeptide concentration of 0.3 μM or higher can be regarded as a standard by which it is judged to be insulin resistant.

Further, in the method according to the present invention, a combination with any other finding related to the condition or disease state of insulin resistance is acceptable.

### [5. Diabetes Marker Analysis Method]

An evaluation of diabetes can be achieved by analyzing the blood concentration of the above-described diabetes marker according to the present invention.

The “evaluation of diabetes” includes identifying the affected state of diabetes, and more specifically includes performing prediction, detection, diagnosis, monitoring, staging, and prognosis of diabetes. The prediction of diabetes includes identifying borderline diabetes (early diagnosis of diabetes). The borderline diabetes includes an insulin-resistance state without hyperglycemia. The prognosis includes, when treatment for diabetes is performed, determining the presence or absence of improvement in diabetes or the degree of the improvement after the treatment.

A diabetes marker analysis method according to the present invention can be performed in the same manner as in the above-described insulin resistance marker analysis method by replacing “insulin resistance” with “diabetes”.

A method for evaluating diabetes according to the present invention includes the steps of: measuring a concentration of the above-described polypeptide in a collected blood sample; and comparing the obtained measured concentration with a normal blood concentration of the above-described polypeptide.

The blood sample is collected from a living body to be evaluated for diabetes. An increase in the measured concentration of the above-described polypeptide in the collected blood sample above the normal blood concentration of the above-described polypeptide can be regarded as one index indicating that the above-described living body is predicted to develop diabetes or has a high possibility of being in a diabetic state.

In the method according to the present invention, the measured result of the polypeptide according to the present invention may be used singly or in combination with any other evaluation criterion related to diabetes (e.g., other diabetes markers, blood glucose level, hemoglobin A1c). Therefore, the method according to the present invention may include measuring a concentration of a substance that is to be another evaluation criterion as well as a concentration of the diabetes marker according to the present invention.

Additionally, in the preparation of a collected blood sample and the measurement of a polypeptide concentration, which are performed in the diabetes marker analysis method according to the present invention, the same method as in the above-described insulin resistance marker analysis method can be used. An evaluation between normal and diabetes may be made based on the same criterion as in the evaluation between normal and insulin resistance in the above-described insulin resistance marker analysis method.

### [6. Method for Screening Substance Improving Insulin Resistance]

The above-described insulin resistance marker according to the present invention is useful in screening a substance improving insulin resistance. The present invention provides a method for screening a substance improving systemic insulin resistance by utilizing the fact that the above-described insulin resistance marker shows association with systemic insulin resistance in a living body. In the screening method according to the present invention, a determination is made as to whether the expression or secretion of the insulin resistance marker according to the present invention is regulated or not by administering a candidate substance to a living body. In such a determination, the blood concentration of the above-described insulin resistance marker according to the present invention in the living body is used as one index.

The candidate substance judged by the screening method according to the present invention to improve insulin resistance is useful as an active ingredient of a pharmaceutical composition for improving systemic insulin resistance. Further, the present inventors have found the relationship between the polypeptide as a component of the insulin resistance marker according to the present invention and obesity. More specifically, the present inventors have found that obesity can be suppressed by suppressing the expression of this polypeptide. That is, the candidate substance found by the screening method according to the present invention is an anti-obesity substance, and a pharmaceutical composition containing, as an active ingredient, the candidate substance is excellent in that it is possible to avoid concerns of obesity as a side effect of a thiazolidine derivative for use in a conventional drug for treating insulin resistance.

The “improvement of insulin resistance” includes changing insulin resistance to higher insulin sensitivity and changing insulin resistance to higher insulin-dependent glucose transport activity, and more specifically includes controlling an insulin signal transduction-suppressing action in insulin resistance and controlling an insulin-dependent glucose transport system-suppressing action, and an example thereof includes treating a disease with insulin resistance.

### [6-1. Processes of Screening Substance Improving Insulin Resistance]

The screening method according to the present invention includes the following steps of:

administering a candidate substance to a non-human animal expressing insulin resistance;

measuring a blood concentration of a polypeptide comprising at least 15 continuous amino acids in an amino acid sequence represented by SEQ ID No. 1 in the non-human animal administered with the candidate substance; and

comparing the measured blood concentration of the polypeptide in the non-human animal administered with the candidate substance, with a blood concentration of the polypeptide in a non-human animal not administered with the candidate substance, wherein

a reduction in the concentration in the non-human animal administered with the candidate substance below the concentration in the non-human animal not administered with the candidate substance is regarded as one index for selecting the candidate substance as a substance improving insulin resistance.

In the method according to the present invention, the measured result of the polypeptide according to the present invention may be used singly or in combination with the measured result of any other marker related to the condition or disease state of insulin resistance. Therefore, the method according to the present invention may include measuring concentrations of other markers as well as a concentration of the polypeptide according to the present invention.

### [6-2. Living Body]

The above-described living body may be a human or a non-human animal. The non-human animal is not particularly limited, but may be a mammal. Examples of the mammal include primates (except for humans), rodents (e.g., mice and rats), rabbits, dogs, cats, pigs, cows, sheep, and horses.

The “living body not administered with the candidate substance” is a living body with development of insulin resistance, and may be a living body before administration of the candidate substance or a living body serving as a negative control. The development of insulin resistance in a living body may be determined by evaluating that the living body is insulin resistant by a known insulin resistance evaluation method or the above-described insulin resistance marker analysis method according to the present invention.

### [6-3. Candidate Substance]

### [6-3-1. Sample Containing Candidate Substance]

Examples of a possible candidate substance include, but are not particularly limited to, nucleic acids, proteins, peptides, organic compounds, and inorganic compounds. Examples of a sample containing such a candidate substance include, but are not particularly limited to, cell extracts, expression products of polynucleotide libraries, synthetic low-molecular compounds, synthetic peptides, natural compounds, and mixtures thereof.

### [6-3-2. Preliminary Screening of Candidate Substance]

As the candidate substance in the screening method according to the present invention, a candidate substance can be used which is obtained by another method for screening a substance improving insulin resistance (hereinafter, referred to as a “preliminary screening method”). An example of such a preliminary screening method includes the method described in JP-A-2009-204475.

More specifically, according to the preliminary screening method, a substance improving insulin resistance is subjected to preliminary screening by:

regulating the expression of an insulin resistance marker (in the case of preliminary screening, an insulin resistance marker includes both the polypeptide according to the present invention and a polynucleotide encoding an amino acid sequence of the polypeptide according to the present invention) or,

controlling the function of the insulin resistance marker (specifically, insulin resistance-inducing activity, e.g., activity of suppressing an insulin signaling pathway).

More specifically, the preliminary method for screening a substance improving insulin resistance based on an expression level of an insulin resistance marker includes the following steps of:

bringing an insulin resistance sample into contact with a candidate substance;

measuring a level or function of an insulin resistance marker in the insulin resistance sample when the insulin resistance sample is brought into contact with the candidate substance; and

comparing the measured level or function of the insulin resistance marker when the insulin resistance sample is brought into contact with the candidate substance, with a level or function of the insulin resistance marker when the insulin resistance sample is not brought into contact with the candidate substance, wherein

a reduction in the measured level or function of the insulin resistance marker when the insulin resistance sample is brought into contact with the candidate sample below the level or function of the insulin resistance marker when the insulin resistance sample is not brought into contact with the candidate substance is regarded as one index for selecting the candidate substance as a substance improving insulin resistance.

The preliminary screening uses, as an index, the level of a polynucleotide encoding the polypeptide according to the present invention (a polynucleotide comprising at least 45 continuous bases in a base sequence represented by SEQ ID No. 2), the level (expression level or secretion level) of a polypeptide as a translated product of the polynucleotide, or the function of the polypeptide (insulin resistance-inducing activity).

Examples of the insulin resistance sample used when the preliminary screening is performed using, as an index, the level of the polynucleotide include an aqueous solution containing the polynucleotide and a fraction derived from cells expressing the polynucleotide (e.g., a cell lysate, a cell homogenate, a nuclear extract). Examples of the cells include cells endogenously expressing the polynucleotide such as mouse cells (e.g., 3T3-L1, NIH3T3) and human cells (e.g., A431, MCF-7). Other examples thereof include transgenic cells obtained by introducing the polynucleotide into host cells such as mouse-derived cells (e.g., NIH 3T3, 0127, COP, MOP, WOP), hamster-derive cells (e.g., CHO, CHO DHFR—), monkey-derived cells (e.g., COS-7, COS-1, CV-1), human-derived cells (e.g., HeLa), and insect-derived cells (e.g., Sf21, Sf9, High Five).

Examples of the insulin resistance sample used when the preliminary screening is performed using, as an index, the level of the polypeptide include an aqueous solution containing the polypeptide and a fraction derived from cells expressing the polynucleotide (e.g., a cell lysate, a cell homogenate, a nuclear extract). As the cells, the same cells as those expressing the polynucleotide can be used. The cells express the polynucleotide and then express the polypeptide as a translated product thereof.

For example, when a cell that requires an expression-inducing substance (e.g., TNFα, glucocorticoid (dexamethasone), free fatty acid, resistin, PAI-1) for expression of the insulin resistance marker is used in the preliminary screening performed using, as an index, the level of the polynucleotide or the polypeptide, selection of a substance can be performed using, as an index, a reduction in the level of the insulin resistance marker in a cell brought into contact with a candidate substance in the presence of the expression-inducing substance below the level of the insulin resistance marker in a cell not brought into contact with the candidate substance in the presence of the expression-inducing substance.

As the insulin resistance sample used when the preliminary screening is performed using, as an index, the function of the polypeptide, a sample in which the insulin resistance marker is constantly present is used, and specific examples thereof include a sample exposed to the insulin resistance marker for a long period of time and a cell in which the insulin resistance marker is constantly expressed or secreted.

An example of the insulin resistance-inducing activity includes activity of suppressing an insulin signaling pathway and an insulin-dependent glucose transport system. The activity refers to, for example, activity of suppressing an increase in phosphorylated Akt mediated by an insulin receptor or of increasing or decreasing another factor that functions downstream from an insulin receptor in an insulin signaling pathway to suppress insulin-dependent glucose transport from outside a cell. These functions are acquired due to the constant presence of the insulin resistance marker. In the preliminary screening, a substance that suppresses the above-described function of the insulin resistance marker is selected.

For example, when a cell that requires an expression-inducing substance (e.g., TNFα, glucocorticoid (dexamethasone), free fatty acid, resistin, PAI-1) for expression of the insulin resistance marker is used in the preliminary screening performed using, as an index, the function of the polypeptide, selection of a candidate substance can be performed using, as an index, a reduction in the level of phosphorylated Atk in an insulin-resistance cell brought into contact with a candidate substance in the presence of the expression-inducing substance below the level of phosphorylated Akt in an insulin-resistance cell not brought into contact with the candidate substance in the presence of the expression-inducing substance.

Among insulin signaling-related factors, in the case of a factor, such as the above-described phosphorylated Akt, that activates an insulin-dependent glucose transport system due to its up-regulated level, selection of a substance that controls the function of the insulin resistance marker can be performed in the same manner as described above.

On the other hand, among insulin signaling-related factors, in the case of a factor, such as phosphorylated Akt, that inactivates an insulin-dependent glucose transport system due to its down-regulated level, unlike the above case, selection of a candidate substance can be performed using, as one index, an increase in the level of the factor in an insulin resistance sample brought into contact with a candidate substance above the level of the factor in an insulin resistance sample not brought into contact with the candidate substance; or an increase in the expression level of the factor expressed in an insulin-resistance cell brought into contact with a candidate substance above the level of the factor in an insulin-resistance cell not brought into contact with the candidate substance.

### [6-3-3. Method for Administering Candidate Substance]

A method for administering a candidate substance is not particularly limited, and any method used in the field of drug screening can be used. Therefore, a candidate substance may be administered either orally (e.g., buccal or sublingual administration) or parenterally (e.g., intravenous, intramuscular, subcutaneous, transdermal, transnasal, or lung administration).

### [6-4. Collected Blood Sample]

In order to measure the blood concentration of the insulin resistance marker, blood can be collected from a living body.

A collected blood sample may be subjected to any pretreatment that does not have essentially influence on analysis. Examples of such pretreatment include anticoagulation treatment and centrifugal separation (plasma separation and serum separation), but plasma exchange, serum exchange, and addition of a substance having the ability to bind to the polypeptide according to the present invention (by which the polypeptide according to the present invention may be eliminated from an analysis system) are excluded. Therefore, examples of the collected blood sample include whole blood, plasma, and serum.

### [6-5. Method for Measuring Polypeptide Concentration]

The blood concentration of the insulin resistance marker can be measured by measuring the concentration of the polypeptide according to the present invention in a collected blood sample.

A method for measuring the concentration of the polypeptide is not particularly limited, and any method capable of specifically detecting a specific polypeptide can be used. Specifically, it may be measured by a test based on biospecific affinity or by mass spectrometry.

The test based on biospecific affinity is a well-known method to those skilled in the art, and examples thereof include, but are not particularly limited to, immunoassays. Specific examples of the immunoassays include competitive and non-competitive assay systems such as Western blotting, radio immunoassay, ELISA, sandwich immunoassay, immunoprecipitation, precipitation reaction, gel diffusion precipitin reaction, immunodiffusion, aggregation measurement, complement fixation analysis, immunoradiometric assay, fluorescent immunoassay, and protein A immunoassay. In these methods, the presence of an antibody that binds to progranulin in a collected blood sample is detected. The testis performed, under the condition that an immunocomplex composed of a polypeptide to be measured and an antibody to the polypeptide can be formed, by bringing a collected blood sample into contact with the antibody. Amore specific protocol can be easily determined by those skilled in the art.

As a method measured by mass spectrometry, a method capable of quantitatively measuring a polypeptide can be appropriately selected by those skilled in the art.

For example, an isotope labeling method can be mentioned. The isotope labeling method is preferred because it is excellent in quantitativity. In this case, the measurement can be performed by determining a difference in the presence of the above-described polypeptide between an appropriate control sample, such as a sample prepared to contain a known level of the above-described polypeptide or a normal sample, and a sample of an individual to be analyzed.

Further, a method can be mentioned, in which an immunoprecipitate is obtained from a collected blood sample and subjected to mass spectrometry (WO 2008/065806). In this case, the polypeptide according to the present invention present in a collected blood sample is immunoprecipitated with a carrier having an antibody to the polypeptide bonded thereto or a carrier having the antibody and a molecule, which can specifically bind to the antibody, bonded thereto. The obtained polypeptide immunoprecipitate is washed with an aqueous solution containing a charge neutralizer (e.g., an ammonium salt such as ammonium carbonate or ammonium hydrogen carbonate). Then, the immunoprecipitate can be directly subjected to mass spectrometry to quantitatively measure the polypeptide based on an internal standard.

### [6-6. Evaluation of Improvement in Insulin Resistance]

The degree of a reduction in the measured blood concentration of the insulin resistance marker varies depending on the measurement method, and is therefore not particularly limited. However, it is preferable to reduce to a concentration close to a normal concentration or to a normal concentration from the viewpoint of curing insulin resistance.

The “normal concentration” includes the blood concentration in an insulin-sensitive living body. The “normal concentration” may be determined by evaluating that it is regarded as normal by a known insulin resistance evaluation method and/or the above-described insulin resistance marker analysis method according to the present invention.

Therefore, the degree of a reduction in the concentration of the polypeptide according to the present invention measured in the screening method according to the present invention by which insulin resistance in a living body is judged to have improved is not particularly limited. The measured concentration of the polypeptide according to the present invention at which insulin resistance is judged to have improved may be reduced to, for example, the concentration of the polypeptide according to the present invention in the blood of a living body which should be judged to fall within a normal range by a known insulin resistance evaluation method and/or the above-described insulin resistance marker analysis method according to the present invention, or the concentration of the polypeptide according to the present invention in the blood of a living body at which insulin resistance should be judged to have improved by administration of an existing drug for treating insulin resistance.

For example, a degree such that the blood concentration of the insulin resistance marker is 1/1.25 or less, preferably 1/1.5 or less, more preferably ½ or less (molar basis) of that in a living body not administered with a candidate substance can be regarded as a standard by which insulin resistance is judged to have improved. For example, in the case of rodents such as mice, a reduction in the measured concentration of the polypeptide to 0.2 μM to 0.3 μM can be regarded as a standard by which insulin resistance is judged to have improved.

Further, in the method according to the present invention, a combination with any other finding related to the condition or disease state of insulin resistance is acceptable.

### [7. Method for Screening Substance Improving Diabetes]

The above-described diabetes marker according to the present invention is useful in screening a substance improving diabetes. The present invention provides a method for screening a substance improving diabetes by utilizing the fact that the above-described diabetes marker shows association with diabetes in a living body. In the screening method according to the present invention, a determination is made as to whether symptoms of diabetes are improved or not by administering a candidate substance to a living body. In such a determination, the blood concentration of the above-described diabetes marker according to the present invention in the living body is used as one index.

The candidate substance judged by the screening method according to the present invention to improve diabetes is useful as an active ingredient of a pharmaceutical composition for improving diabetes. Further, the present inventors have found the relationship between the polypeptide as a component of the diabetes marker according to the present invention and obesity. More specifically, the present inventors have found that obesity can be suppressed by suppressing the expression of this polypeptide. That is, the candidate substance found by the screening method according to the present invention is an anti-obesity substance, and a pharmaceutical composition containing, as an active ingredient, the candidate substance is excellent in that it is possible to avoid concerns of obesity as a side effect of a thiazolidine derivative for use in a conventional drug for treating diabetes.

The “improvement of diabetes” includes changing insulin resistance as one of symptoms of diabetes to higher insulin sensitivity and changing insulin resistance to higher insulin sensitivity and changing insulin resistance to higher insulin-dependent glucose transport activity, and more specifically includes controlling an insulin signal transduction-suppressing action in insulin resistance and controlling an insulin-dependent glucose transport system-suppressing action. Further, a reduction of a high blood glucose level as one of symptoms of diabetes is also included.

The method for screening a substance improving diabetes according to the present invention can be performed in the same manner as in the above-described method for screening a substance improving insulin resistance by replacing “insulin resistance” with “diabetes”.

The screening method according to the present invention includes the following steps of:

administering a candidate substance to a living body suffering from diabetes;

measuring a blood concentration of a polypeptide comprising at least 15 continuous amino acids in an amino acid sequence represented by SEQ ID No. 1 in the living body administered with the candidate substance; and

comparing the measured blood concentration of the polypeptide in the living body administered with the candidate substance, with a blood concentration of the polypeptide in a living body not administered with the candidate substance, wherein

a reduction in the measured concentration in the living body administered with the candidate substance below the concentration in the living body not administered with the candidate substance is regarded as one index for selecting the candidate substance as a substance improving or suppressing diabetes.

In the method according to the present invention, the measured result of the polypeptide according to the present invention may be used singly or in combination with any other evaluation criterion related to diabetes (e.g., other diabetes markers, blood glucose level, hemoglobin Alc). Therefore, the method according to the present invention may include measuring a concentration of a substance that is to be another evaluation criterion as well as a concentration of the polypeptide according to the present invention.

Additionally, the living body and the candidate substance used in the screening may be the same as those used in the above-described method for screening a substance improving insulin resistance. The preparation of a collected blood sample, the administration of a candidate substance, and the measurement of a polypeptide concentration can be performed in the same manner as in the above-described method for screening a substance improving insulin resistance.

An evaluation of improvement in diabetes may be made based on the same criterion as used in the evaluation method in the above-described method for screening a substance improving insulin resistance.

### [8. Pharmaceutical Composition for Improvement, Etc., of Systemic Insulin Resistance]

A pharmaceutical composition according to the present invention may be used as a diagnostic drug for evaluating systemic insulin resistance or a potential or practical therapeutic drug for improving systemic insulin resistance.

The insulin resistance marker according to the present invention and a substance selected by the above-described screening method are useful as active ingredients of the pharmaceutical composition.

That is, examples of the active ingredient of the pharmaceutical composition according to the present invention include the following. The pharmaceutical composition can be prepared by mixing a pharmaceutically-acceptable diluent, carrier, excipient, and the like with such an active ingredient.

[8-1. Polypeptide Comprising at Least 15 Continuous Amino Acids in Amino Acid Sequence Represented by SEQ ID No. 1 or Substance Having Structure Similar to that of Polypeptide Comprising at Least 15 Continuous Amino Acids in Amino Acid Sequence Represented by SEQ ID No. 1]

A polypeptide comprising at least 15 continuous amino acids in an amino acid sequence represented by SEQ ID No. 1 which is the insulin resistance marker according to the present invention or a substance having a structure similar to that of the polypeptide has a potential to significantly inhibit the interaction with a protein or receptor that can be bound to the insulin resistance marker. Therefore, the peptide or the substance has a potential to reduce the function of the insulin resistance marker.

Therefore, a pharmaceutical composition containing the polypeptide or the substance as an active ingredient is useful as a therapeutic drug. It is to be noted that the substance having a structure similar to that of the polypeptide may have a structure that produces an action of reducing the interaction with a protein or receptor that can be bound to the insulin resistance marker like the above-mentioned polypeptide, and a specific structural similarity can be appropriately determined by those skilled in the art as long as according to such a viewpoint.

### [8-2. Antibody Against Polypeptide Comprising at Least 15 Continuous Amino Acids in Amino Acid Sequence Represented by SEQ ID No. 1]

The “antibody” includes a polyclonal antibody, a monoclonal antibody, and an antibody prepared by a molecular biological technique. Preparation of these antibodies is performed by a method well known to those skilled in the art.

The “antibody” broadly refers to a substance that immunospecifically binds, and also includes an antibody fragment and an antibody fusion protein.

As has been described above with reference to the insulin resistance marker analysis method, an antibody to a polypeptide comprising at least 15 continuous amino acids in an amino acid sequence represented by SEQ ID No. 1 which is the insulin resistance marker according to the present invention is useful for evaluating systemic insulin resistance. Therefore, a pharmaceutical composition containing the polypeptide as an active ingredient is useful as a diagnostic drug.

The antibody specifically binds to the polypeptide comprising at least 15 continuous amino acids in an amino acid sequence represented by SEQ ID No. 1 which is the insulin resistance marker according to the present invention, and therefore can reduce the function of the insulin resistance marker. Therefore, a pharmaceutical composition containing an antibody against the polypeptide as an active ingredient is useful as a therapeutic drug.

### [8-3. Substance Regulating Expression Level or Secretion Level of Polypeptide Comprising at Least 15 Continuous Amino Acids in Amino Acid Sequence Represented by SEQ ID No. 1]

This substance is selected through preliminary screening in the above-described method for screening a substance improving insulin resistance, and reduces the expression level or secretion level of a polypeptide comprising at least 15 continuous amino acids in an amino acid sequence represented by SEQ ID No. 1 which is the insulin resistance marker according to the present invention. Therefore, a pharmaceutical composition containing this substance as an active ingredient is useful as a therapeutic drug.

### [8-4. Substance Controlling Function of Polypeptide Comprising at Least 15 Continuous Amino Acids in Amino Acid Sequence Represented by SEQ ID No. 1]

This substance is selected through preliminary screening in the above-described method for screening a substance improving insulin resistance, and reduces the function of a polypeptide comprising at least 15 continuous amino acids in an amino acid sequence represented by SEQ ID No. 1 which is the insulin resistance marker according to the present invention. Therefore, a pharmaceutical composition containing this substance as an active ingredient is useful as a therapeutic drug.

Example of such a substance include substances judged by the preliminary screening to increase a substance that regulates an insulin signaling-related factor down-regulated by expression or secretion of the polypeptide that is the insulin resistance marker according to the present invention or to reduce a substance that regulates an insulin signaling-related factor up-regulated by expression or section of the polypeptide that is the insulin resistance marker according to the present invention.

[8-5. Polynucleotide Selected from Group Consisting of Polynucleotide Comprising at Least 45 Continuous Bases in Base Sequence Represented by SEQ ID No. 2 and Polynucleotide Complementary to the Polynucleotide]

The “base sequence represented by SEQ ID No. 2” is a base sequence encoding the polypeptide represented by SEQ ID No. 1, that is, a base sequence encoding a human homolog of progranulin.

The “polynucleotide” includes an oligonucleotide and a polynucleotide. Further, the “polynucleotide” includes DNA and RNA. The DNA includes cDNA, genomic DNA, and synthetic DNA. The RNA includes total RNA, mRNA, rRNA, and synthetic RNA. Further, the “polynucleotide” includes a single-stranded polynucleotide and a double-stranded polynucleotide.

The “polynucleotide comprising at least 45 continuous bases in a base sequence represented by SEQ ID No. 2” includes progranulin, granulin peptides (specifically, 1-granulin, 2-granulin, 3-granulin, 4-granulin, 5-granulin, 6-granulin, and 7-granulin), and a polynucleotide encoding any polypeptide containing a minimum sequence involved in insulin resistance in a progranulin sequence in its part or entirety.

The polynucleotide comprising at least 45 continuous bases in a base sequence represented by SEQ ID No. 2 may be a polynucleotide comprising at least 150 continuous bases in the sequence or a polynucleotide comprising at least 1779 continuous bases in the sequence.

The “polynucleotide comprising at least 45 continuous bases in a base sequence represented by SEQ ID No. 2” includes a polynucleotide specified by a sequence comprising at least 45 continuous bases in a base sequence represented by SEQ ID No. 2 and polynucleotides encoding polypeptides that are homologs and mutants of a polypeptide encoded by the polynucleotide and have an equivalent biological function involved in insulin resistance to that of the polypeptide.

The polynucleotides encoding the homologs include the complete or partial sequences of homologs of other species corresponding to a human homolog of a polynucleotide encoding progranulin. Examples of the other species include mammals other than humans, and specific examples thereof include primates, rodents (e.g., mice and rats), rabbits, dogs, cats, pigs, cows, sheep, and horses.

Therefore, the “polynucleotide selected from the group consisting of a polynucleotide comprising at least 45 continuous bases in a base sequence represented by SEQ ID No. 2 and a polynucleotide complementary to the polynucleotide” includes a polynucleotide selected from the group consisting of a polynucleotide comprising at least 45 continuous bases in a base sequence represented by SEQ ID No. 4 which is a mouse homolog and a polynucleotide complementary to the polynucleotide.

The polynucleotides encoding the mutants include naturally-occurring mutants of a polynucleotide encoding progranulin and mutants modified by artificial replacement, addition, insertion, and deletion of a base.

Examples of these polynucleotides encoding the homologs and the mutants include those having a homology of at least 80%, preferably at least 85%, more preferably at least 90%, even more preferably at least 95% with a sequence comprising at least 45 continuous bases in a base sequence represented by SEQ ID No. 2.

When the above-described polynucleotide is target RNA, double-stranded RNA having a sequence homology with the target RNA (this is also included in the polynucleotide provided as the insulin resistance marker according to the present invention) can induce dissociation of the target RNA by an RNA interference mechanism. Therefore, a pharmaceutical composition containing the polynucleotide as an active ingredient is useful as a therapeutic drug.

Further, when the above-described polynucleotide is a target, a polynucleotide complementary to the target polynucleotide can inhibit the activation of a gene when administered. Therefore, a pharmaceutical composition containing the polynucleotide as an active ingredient is useful as a therapeutic drug.

[8-6. Substance Regulating Expression Level of Polynucleotide Selected from Group Consisting of Polynucleotide Comprising at Least 45 Continuous Bases in Base Sequence Represented by SEQ ID No. 2 and Polynucleotide Complementary to the Polynucleotide]

This substance is selected through preliminary screening in the above-described method for screening a substance improving insulin resistance, and reduces the expression level of a polynucleotide selected from the group consisting of a polynucleotide comprising at least 45 continuous bases in the above-described base sequence represented by SEQ ID No. 2 and a polynucleotide complementary to the polynucleotide. Therefore, a pharmaceutical composition containing this substance as an active ingredient is useful as a therapeutic drug.

### [9. Pharmaceutical Composition for Improvement, Etc., of Diabetes]

A pharmaceutical composition in the present invention may be used as a diagnostic drug for evaluating diabetes or a potential or practical therapeutic drug for improving diabetes. As this pharmaceutical composition, the same pharmaceutical compositions as described above with reference to the pharmaceutical composition for improvement, etc., of insulin resistance can be mentioned by replacing “insulin resistance” with “diabetes”.

### [10. Method for Improving Insulin Resistance or Diabetes]

The concentration of the insulin resistance marker or the diabetes marker in a living body can be reduced by administering the above-described pharmaceutical composition to the living body or by infecting the living body with the above-described polynucleotide or a virus having the above-described polynucleotide introduced thereinto. This makes it possible to improve insulin resistance or diabetes in the living body.

### EXAMPLES

Hereinbelow, the present invention will be described in more detail with reference to examples. However, the present invention is not limited to these examples. In the following description, progranulin refers to full-length progranulin.

### Reference Example 1

### Verification of Progranulin Expression in Adipose Tissues of Mice (ob/ob) that Spontaneously Develop Insulin Resistance

An ob/ob mouse is an appropriate model animal for insulin resistance which is genetically obese and shows significant insulin resistance. More specifically, an ob/ob mouse shows hyperinsulinemia, hypercortisolemia, hyperglycemia, insulin resistance, changes in central nerve system, overeating, reduction in the metabolic rate of brown adipose cells, increase in the weight of white adipose cells, reduction in fertility etc.

On the other hand, an ob/+ mouse as a heterozygote does not present with obesity and insulin resistance.

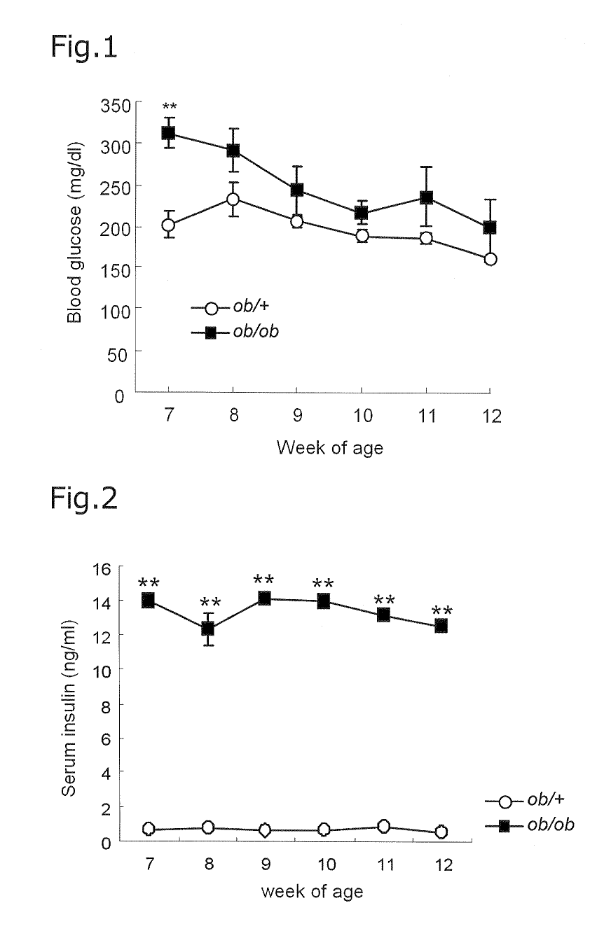

Changes in blood glucose and blood insulin concentration in ob/ob mice and ob/+ mice are shown in FIGS. 1 and 2, respectively. In FIG. 1, the horizontal axis represents weeks of age (Week of age) and the vertical axis represents blood glucose (Blood glucose) (mg/dL). The ob/ob mice are slightly higher in blood glucose than the ob/+ mice, but the difference between them is not significant (as compared to that between diabetes model mice db/db and control mice db/+ described later with reference to Example 2). In FIG. 2, the horizontal axis represents weeks of age (Week of age) and the vertical axis represents serum insulin concentration (Serum insulin) (ng/mL) The ob/ob mice show significantly higher insulin resistance than the ob/+ mice.

Adipose tissues (mesenteric fat tissue (MF), epididymal fat tissue (EF), and subcutaneous adipose tissue (SC)) were extracted from seven-week-old ob/ob mice and ob/+ mice and subjected to real-time PCR analysis to determine the expression levels of mRNA for progranulin (PGRN).

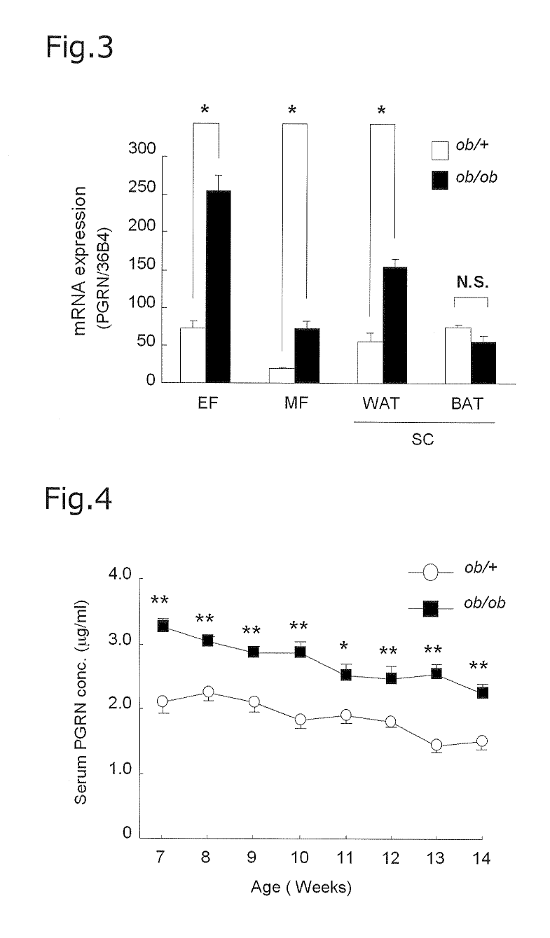

As a result, in the insulin-resistance mice (ob/ob mice), the expression level in epididymal fat tissue (EF) was about 3.52±0.30 (mean±S.D., n=5, p<0.01) times, that of mesenteric fat tissue (MF) was about 3.91±0.50 (mean±S.D., n=5, p<0.01) times, and that of subcutaneous adipose tissue (SC) (white adipose tissue (WAT)) was about 3.08±0.16 (mean±S.D., n=5, p<0.01) times higher than those of the non-insulin-resistance (insulin sensitive) mice (ob/+ mice) as a control group. The results are shown in FIG. 3. In FIG. 3, the horizontal axis represents the type of adipose tissue (epididymal fat tissue (EF), mesenteric fat tissue (MF), and subcutaneous adipose tissue (SC) (white adipose tissue (WAT) and brown adipose tissue (BAT)), and the vertical axis represents the expression level of mRNA for progranulin (PGRN) (mRNA expression (PGRN/36B4)) compared with that of the control group.

### Example 1

### Verification of Blood Progranulin Concentrations in Mice (ob/ob) Having Obesity and Spontaneously Developing Insulin Resistance

Blood samples were collected from seven-week-old ob/ob mice and ob/+ mice, allowed to stand at room temperature, and centrifuged to prepare serum samples. Serum progranulin concentrations were measured by ELISA. The serum progranulin concentration of the ob/+ mice was 2.09±0.17 (mean±S.D, n 5) μg/mL and that of the ob/ob mice was 3.21±0.16 (mean±S.D., n=5) μg/mL, and thus, serum progranulin concentrations were significantly (p<0.01) higher in diabetes.

Changes in blood progranulin concentrations in the ob/ob mice and the ob/+ mice are shown in FIG. 4. In FIG. 4, the horizontal axis represents weeks of age (Age (Weeks)) and the vertical axis represents serum progranulin concentration (Serum PGRN conc.) (μg/mL).

From Reference Example 1 and Example 1, it was demonstrated that the blood concentration of progranulin was associated with insulin resistance.

### Example 2

### Verification of Blood Progranulin Concentrations in Mice (db/db) Having Obesity and Spontaneously Developing Diabetes

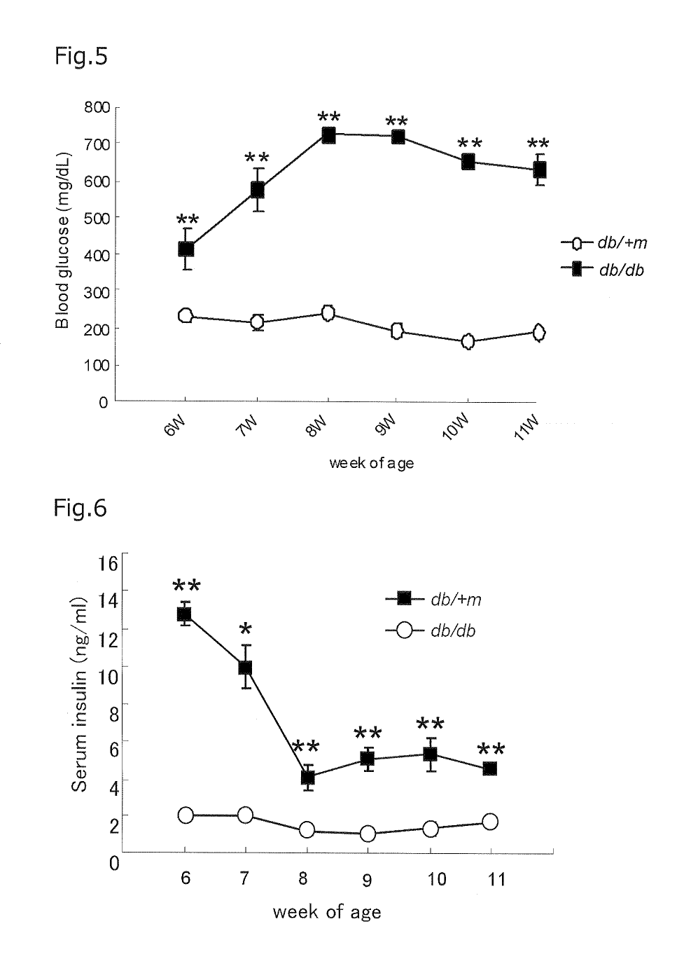

A db/db mouse is an appropriate model mouse for diabetes which is genetically obese and shows significant hyperglycemia. Changes in blood glucose and blood insulin concentration in db/db mice and control model mice (db/+m) are shown in FIGS. 5 and 6, respectively. In FIG. 5, the horizontal axis represents weeks of age (Week of age) and the vertical axis represents blood glucose (Blood glucose) (mg/dL). The db/db mice show a rapid increase in blood glucose shortly after birth and are significantly more hyperglycemic than the db/+ mice. In FIG. 6, the horizontal axis represents weeks of age (Week of age) and the vertical axis represents serum insulin concentration (Serum insulin) (ng/mL). The db/db mice have extremely high blood insulin concentrations at a young age (6 to 8 weeks old) but show a reduction in blood insulin concentration with increasing blood glucose level shown in FIG. 5. The blood insulin concentration can serve as an indirect index for insulin resistance (i.e., the db/db mice show insulin resistance throughout their lives), but the db/db mice have relatively strong insulin resistance at 6 to 8 weeks old, which suggests that they are in the early stage of development of diabetes.

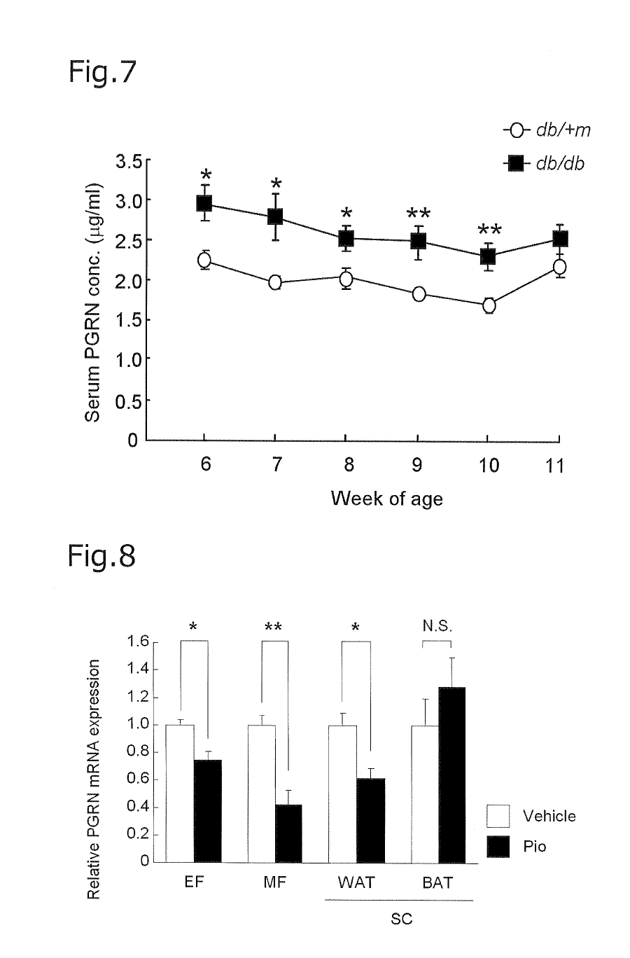

Changes in blood progranulin concentrations in the model mice (db/db) that spontaneously develop genetic diabetes and the control model mice (db/+m) are shown in FIG. 7. In FIG. 7, the horizontal axis represents weeks of age (Week of age) and the vertical axis represents serum progranulin concentration (Serum PGRN conc.) (μg/mL). As shown in FIG. 7, the db/db mice have higher progranulin concentrations than the db/+ mice in insulin resistance, but after development of diabetes (i.e., after an increase in blood glucose), the difference in progranulin concentration between the db/db mice and the db/m+ mice is reduced due to a reduction in insulin resistance. However, the db/db mice stably have high progranulin concentrations throughout their lives.

From Example 2, it was demonstrated that the blood concentration of progranulin was specific to insulin resistance and was associated with diabetes.

### Example 3

### Verification of Progranulin Expression in Adipose Tissues and Blood Progranulin Concentrations in Spontaneously Diabetic Model Mice (ob/ob) Orally Administered with Thiazolidine Derivative

Ten-week-old ob/ob mice were orally administered with 30 mg/kg of a thiazolidine derivative (pioglitazone) every day for 1 week. An oral glucose tolerance test (OGTT) was performed on these mice and a control group (Vehicle) administered with only a solvent (10% (w/v) carboxymethyl cellulose). As a result, it has been reported that the pioglitazone administration group had significantly low casual blood glucose levels (at 0 min) and blood glucose levels after a glucose load, that is, impaired glucose tolerance had been improved (Non-Patent Document 6: Journal of Biological Chemistry 281, 8748-8755).

In this case, when the expression levels of mRNA for progranulin in adipose tissues of the pioglitazone administration group (Pio) were measured and compared with those of the control group (Vehicle), the expression level in mesenteric fat tissue (MF) of the pioglitazone administration group was 0.42±0.11 (mean±S.D., n: pioglitazone administration group=5, control group=4, p<0.01) times that of the control group, the expression level in epididymal fat tissue (EF) of the pioglitazone administration group was 0.74±0.06 (mean±S.D., n: pioglitazone administration group=5, control group=4, p<0.05) times that of the control group, and the expression level in subcutaneous adipose tissue (SC) (white adipose tissue (WAT)) of the pioglitazone administration group was 0.61±0.07 (mean±S.D., n: pioglitazone administration group=5, control group=4, p<0.05) times that of the control group, and the expression levels of mRNA of the pioglitazone administration group were significantly lower. The results are shown in FIG. 8. In FIG. 8, the horizontal axis represents the type of adipose tissue (epididymal fat tissue (EF), mesenteric fat tissue (MF), subcutaneous adipose tissue (SC) (white adipose tissue (WAT) and brown adipose tissue (BAT)), the vertical axis represents the relative expression level of progranulin mRNA (relative PRGN mRNA expression), “Vehicle” represents the control group, and “Pio” represents the pioglitazone administration group.

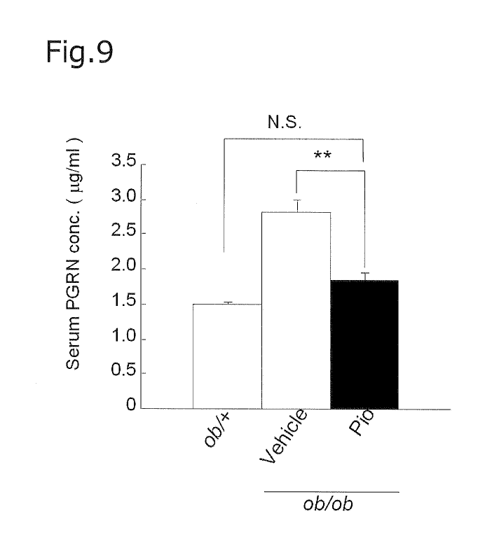

Further, when the serum progranulin concentration was measured, the serum progranulin concentration of the control group (Vehicle) was 2.57±0.28 (mean±S.D., n=4) μg/mL, and that of the pioglitazone administration group (Pio) was 1.86±0.10 (mean±S.D., n=5) μg/mL, and thus, the serum progranulin concentrations of the pioglitazone administration group were significantly (p<0.05) lower. The results are shown in FIG. 9. In FIG. 9, the horizontal axis represents the type of group to be measured (pioglitazone administration group (Pio), control group (Vehicle), and ob/+ mice) and the vertical axis represents the serum progranulin concentration (Serum PGRN conc.) (μg/mL).

### Reference Example 2

### Analysis of Mice Lacking Progranulin Gene

In order to elucidate the causal relationship between the expression of progranulin and obesity-induced insulin resistance, knock-out mice lacking a progranulin gene were analyzed. Specifically, knock-out mice lacking a progranulin gene (PGRN−/−) (Behavioural Brain Research 185 (2007) 110-118) bred at the Riken BioResource Center were obtained. The PGRN−/− mice and wild-type mice (WT) having a progranulin gene as a control group were bred with feeding a standard diet (normal feed CE-2 (manufactured by CLEA Japan, Inc.): lipid 16 kcal %) and a high-fat diet (D12492 feed (manufactured by Research Diets, Inc.): lipid 60 kcal %)

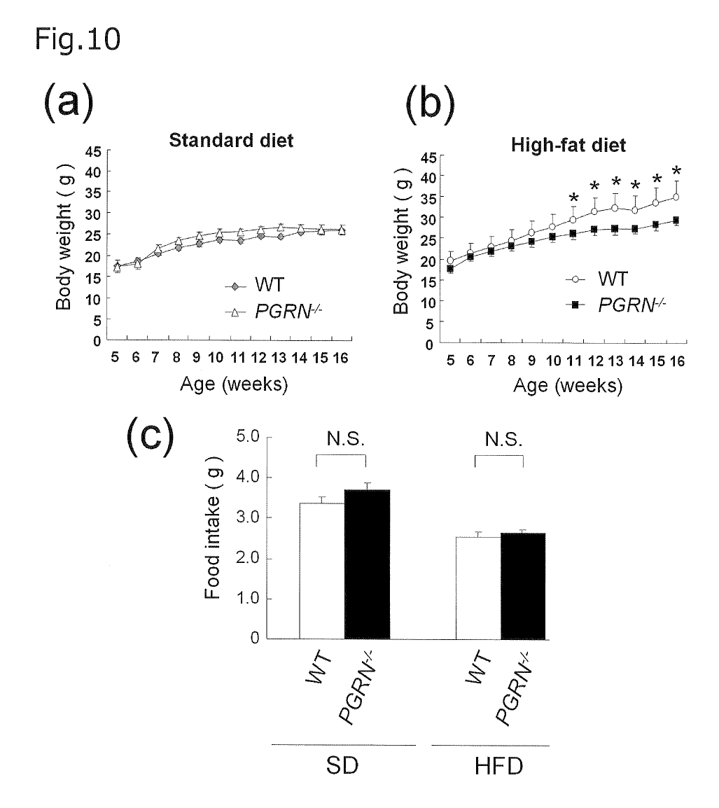

Changes in the body weight of the knock-out mice (PGRN−/−) and the wild-type mice (WT) during breeding are shown in FIG. 10 (a) (in the case of feeding standard diet) and FIG. 10 (b) (in the case of feeding high-fat diet). In FIGS. 10 (a) and 10 (b), the horizontal axes represent the weeks of age (Week of age) and the vertical axes represent the body weight (Body weight (g)). FIG. 10(c) shows the food intake of the knock-out mice (PGRN−/−) and the wild-type mice (WT) during breeding. In FIG. 10 (c), the horizontal axis represents the type of group to be measured and the vertical axis represents the food intake (Food intake (g)). As shown in FIG. 10, when the wile-type mice and the PGRN−/− mice were bred with feeding the standard diet (SD), there was no significant difference in body weight (FIG. 10 (a)). On the other hand, when the wild-type mice and the PGRN−/− mice were bred with feeding the high-fat diet (HFD), irrespective of no difference in food intake (FIG. 10 (c)), the body weights of the PGRN−/− mice were lower by about 20% than those of the wild-type mice after the sixth week from the feeding of high-fat diet (FIG. 10 (b))

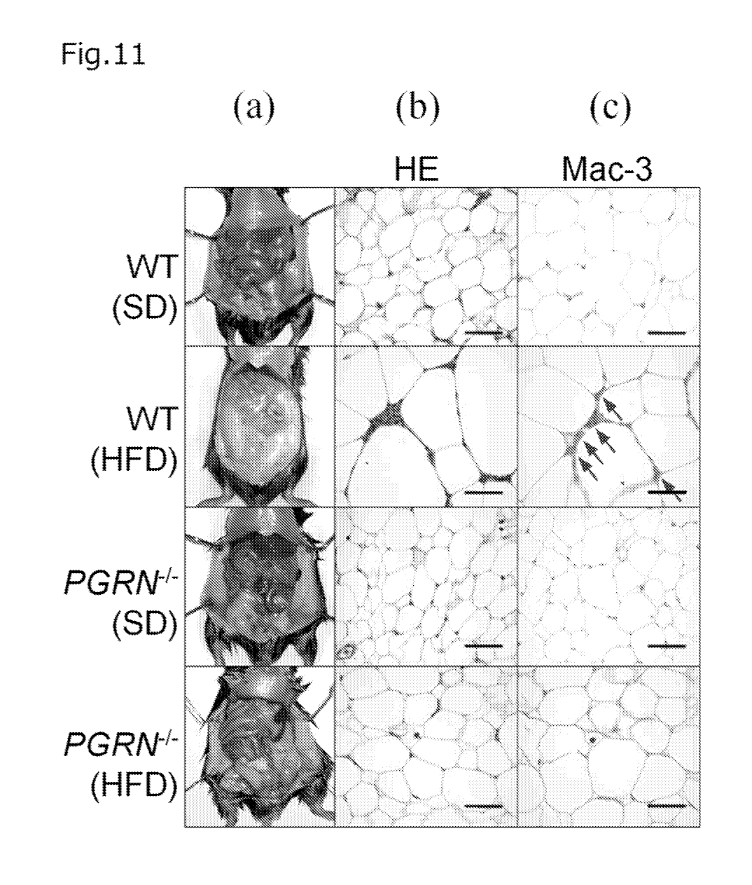

Further, visceral adipose accumulation in wild-type mice (WT) and knock-out mice (PGRN−/−) bred with feeding high-fat diet for 12 weeks was examined (FIG. 11 (a)). Further, the shape of adipose cells was observed by hematoxylin-eosin staining (HE) (FIG. 11 (b)), and inflammatory cells were observed by immunohistochemical staining (Mac3) using antiMac3 antibody (FIG. 11(c)). As shown in FIG. 11(a), significant accumulation of adipose tissues was observed in the wild-type mice bred with feeding high-fat diet, whereas visceral adipose accumulation was not observed in the knock-out mice bred under the same conditions. Further, as shown in FIG. 11(b), significant enlargement of adipose cells was observed in the wild-type mice bred with feeding high-fat diet, whereas enlargement of visceral adipose was not observed in the knock-out mice bred under the same conditions. Further, as shown in FIG. 11(c), significant infiltration of inflammatory cells (indicated by arrows) was observed in the wild-type mice bred with feeding high-fat diet, whereas infiltration of inflammatory cells was not observed in the knock-out mice bred under the same conditions.

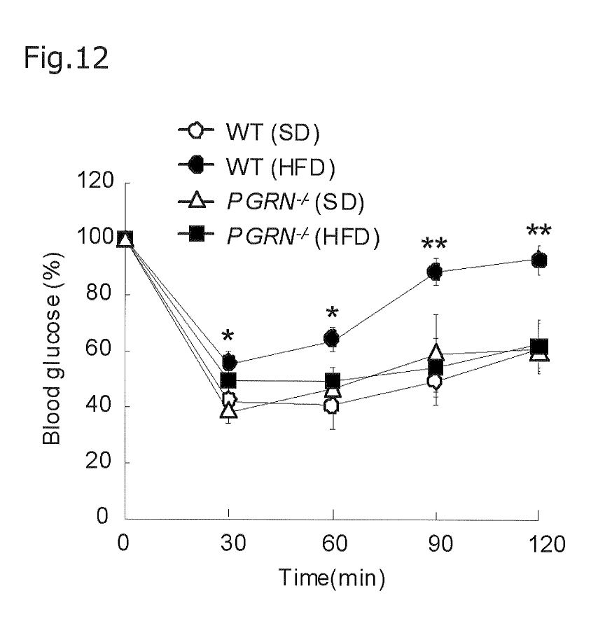

Further, an insulin tolerance test (ITT) was performed on 13-week-old wild-type mice (WT) and knock-out mice (PGRN−/−) bred with feeding high-fat diet (HFD) and 13-week-old wild-type mice (WT) and knock-out mice (PGRN−/−) bred with feeding standard diet (SD) to examine changes in blood glucose after administration of a certain amount of insulin (0.75 U/kg) to abdominal cavity, that is, to examine insulin responsiveness. The results are shown in FIG. 12. In FIG. 12, the horizontal axis represents time (Time (min)) elapsed since administration of insulin and the vertical axis represents relative blood glucose (Blood glucose) (%) when a blood glucose level at the time of insulin injection was defined as 100. As shown in FIG. 12, only the mild-type mice (WT) bred with feeding high-fat diet (HFD) had significantly high blood glucose levels after insulin administration, that is, showed significant insulin resistance. On the other hand, the knock-out mice (PGRN−/−) had significantly low blood glucose levels after insulin administration even when they had been bred with feeding a high-fat diet (HFD), that is, showed good insulin responsiveness.

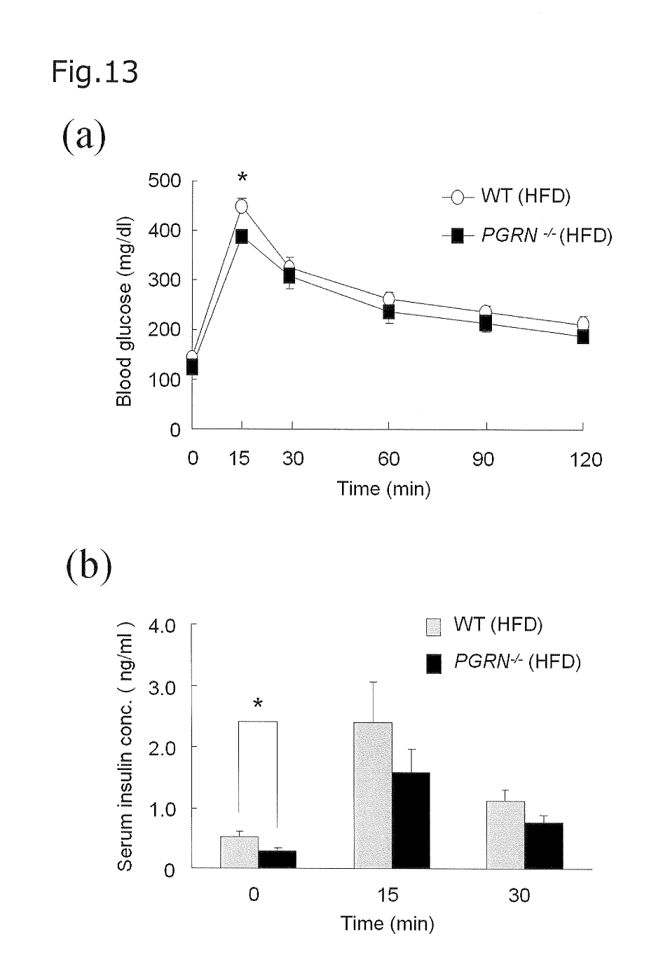

Further, an oral glucose tolerance test (OGTT) was performed on 17-week-old wild-type mice (WT) and knock-out mice (PGRN−/−) bred with feeding high-fat diet (HFD) for 3 months. OGTT is a method for examining changes in blood glucose level and blood insulin concentration after oral administration of a certain amount of glucose to evaluate diabetes. The dose of glucose was 1.5 g/kg. The results are shown in FIG. 13. In FIG. 13(a), the horizontal axis represents time (Time (min)) elapsed since oral administration of glucose, and the vertical axis represents blood glucose level (Blood glucose (mg/mL)) after oral administration of glucose. In FIG. 13(b), the horizontal axis represents time (Time (min)) elapsed since oral administration of glucose, and the vertical axis represents blood insulin concentration (Serum insulin cone.) (ng/mL) after oral administration of glucose. As shown in FIG. 13(a), it was found that the knock-out mice (PGRN−/−) had significantly (p<0.05) lower blood glucose levels at 15 mm, which were highest after glucose administration, than the wild-type mice (WT). Further, as shown in FIG. 13(b), the fasting blood insulin concentrations (at 0 min) of the knock-out mice (PGRN−/−) were significantly lower than those of the wild-type mice (WT). That is, the results suggest that induction of obesity and insulin resistance by a high-fat diet and a reduction in glucose tolerance are completely suppressed in the knock-out mice.

From the above findings, it is considered that obesity can be suppressed by suppressing the expression of a progranulin gene and a progranulin protein and suppressing the function of progranulin and diabetes can be improved by making insulin responsiveness favorable. That is, it is possible to provide a method for improving and suppressing insulin resistance and diabetes which is useful for prevention and treatment of insulin resistance and diabetes without obesity. Further, it is possible to provide a method for screening a substance that improves insulin resistance without obesity and a substance that improves and suppresses diabetes without obesity.

1. A blood insulin resistance marker comprising a polypeptide comprising at least 15 continuous amino acids in an amino acid sequence represented by SEQ ID No. 1.

2. A method for analyzing an insulin resistance marker in a collected blood sample, comprising the steps of:
measuring a concentration of a polypeptide comprising at least 15 continuous amino acids in an amino acid sequence represented by SEQ ID No. 1 in a collected blood sample; and
comparing the obtained measured concentration with a normal blood concentration of the polypeptide.

3. A method for screening a substance improving insulin resistance, comprising the steps of:
administering a candidate substance to a non-human animal showing abnormal insulin sensitivity;
measuring a blood concentration of a polypeptide comprising at least 15 continuous amino acids in an amino acid sequence represented by SEQ ID No. 1 in the non-human animal administered with the candidate substance; and
comparing the measured blood concentration of the polypeptide in the non-human animal administered with the candidate substance, with a blood concentration of the polypeptide in a non-human animal not administered with the candidate substance, wherein
a reduction in the concentration in the non-human animal administered with the candidate substance below the concentration in the non-human animal not administered with the candidate substance is regarded as one index for selecting the candidate substance as a substance improving insulin resistance.

4. A blood diabetes marker comprising a polypeptide comprising at least 15 continuous amino acids in an amino acid sequence represented by SEQ ID No.

5. A method for analyzing a diabetes marker in a collected blood sample, comprising the steps of:
measuring a concentration of a polypeptide comprising at least 15 continuous amino acids in an amino acid sequence represented by SEQ ID No. 1 in a collected blood sample; and
comparing the obtained measured concentration and a normal blood concentration of the polypeptide.

6. A method for screening a substance improving or suppressing diabetes, comprising the steps of:
administering a candidate substance to a non-human animal suffering from diabetes;
measuring a blood concentration of a polypeptide comprising at least 15 continuous amino acids in an amino acid sequence represented by SEQ ID No. 1 in the non-human animal administered with the candidate substance; and
comparing the measured blood concentration of the polypeptide in the non-human animal administered with the candidate substance, with a blood concentration of the polypeptide in a non-human animal not administered with the candidate substance, wherein
a reduction in the concentration in the non-human animal administered with the candidate substance below the concentration in the non-human animal not administered with the candidate substance is regarded as one index for selecting the candidate substance as a substance improving or suppressing diabetes.
